(19) United States
(12) Patent Application Publication
(10) Pub. No.: US 2026/0105666 A1
(43) Pub. Date: Apr. 16, 2026

(54) MULTIPLE FOCAL SPOTS FOR HIGH RESOLUTION COMPUTED TOMOGRAPHY

(71) Applicants: THE JOHNS HOPKINS UNIVERSITY, Baltimore, MD (US); THE TRUSTEES OF THE UNIVERSITY OF PENNSYLVANIA, Philadelphia, PA (US)

(72) Inventors: Joseph Webster STAYMAN, Baltimore, MD (US); Jianan GANG, Baltimore, MD (US); Peter B. NOEL, Bayside, NY (US)

(21) Appl. No.: 19/115,711

(22) PCT Filed: Sep. 28, 2023
(86) PCT No.: PCT/US2023/034000

§ 371 Date: Mar. 26, 2025

Related U.S. Application Data
(60) Provisional application No. 63/377,474, filed on Sep. 28, 2022.

(51) Int. Cl. G06T 12/20; G06T 5/50; G06T 5/70; G16H 30/20

(52) U.S. Cl. CPC G06T 12/20; G06T 5/50; G06T 5/70; G16H 30/20; G06T 2207/10081; G06T 2207/20182; G06T 2207/20221

(57) ABSTRACT

A method for producing high resolution computed tomography (CT) images is disclosed. The method includes combining multiple focal spots in a single data acquisition to provide high-resolution multi-resolution data that is able to resolve fine features and with fluence to reduce noise levels, wherein the multiple focal spots have different focal spot types; and processing the high-resolution multi-resolution data using a model-based iterative reconstruction (MBIR) method.

### CROSS-REFERENCE TO RELATED APPLICATIONS

This application is the national stage entry of International Patent Application No. PCT/US2023/034000, filed on Sep. 28, 2023, and published as WO 2024/072979 A1 on Apr. 4, 2024, which claims the benefit of U.S. provisional application Ser. No. 63/377,474, filed on Sep. 28, 2022, which are hereby incorporated by reference in their entireties.

### FIELD

The present disclosure is directed to high resolution computed tomography (CT), and in particular, to systems and methods for multiple focal spots for high resolution CT.

### BACKGROUND

High spatial resolution CT has enabled a variety of clinical applications. However, further improvements resolution are hampered by current x-ray tubes. Smaller focal spots contribute less focal spot blur but have limited fluence—leading to high levels of noise in CT reconstructions.

Increased spatial resolution in x-ray computed tomography (CT) has enabled improved diagnostics in a number of areas including identification of small fractures, temporal bone imaging, and visualization of fine structures and textures within organs (e.g., lungs, trabecular bone, etc.). The spatial resolution of a CT scanner is limited by several factors including the detector characteristics (scintillator blur, pixel size, etc.), size of the x-ray source focal spot, and motion (both patient and gantry motion within a detector integration period).

Recent innovations have sought resolution improvements—largely through smaller detector pixels that permit collection of higher resolution projection data. However, smaller pixels means fewer x-rays per measurement for a given exposure. While recent systems that use photon-counting detectors have some advantage with lower-count data and the elimination of readout noise, there is still a trade-off between high-resolution imaging capabilities and the delivered x-ray fluence. Specifically, increasing spatial resolution will require increased x-ray exposures.

Thus, one of the fundamental challenges of high-resolution CT is how to deliver sufficient fluence to obtain low noise reconstructions. X-ray tube exposures are limited within specific power constraints. Too much power and one can physically melt or damage the x-ray anode. While various strategies are used to increase the power that may be used (including rotating anodes, sophisticated cooling technology, and focal spot motion) there is generally a trade-off between the power limit and the focal spot size. (Distributing the electron beam over a larger area limits the power to any given point on the target anode.) However, larger focal spots will increase the effects of x-ray source blur and consequently limit spatial resolution. Thus, despite advances in detector technologies, many CT systems are limited in high-resolution due to the x-ray source.

Various strategies have been investigated to overcome these source limitations. While there has been some work in x-ray sources with fine focal spots and higher power requirements (e.g., liquid metal source), such systems have not seen translation to clinical devices. Many algorithmic approaches have been applied in an attempt to regain spatial resolution and limit noise in high-resolution reconstructions. Methods have included deconvolution of source blur, explicit modeling of source blur within a model-based reconstruction approach, blind deconvolution/super-resolution methods, etc. While all of these tools have potential to extend the range of high-resolution CT, they do not directly address current data acquisition limits.

### SUMMARY

According to examples of the present disclosure, a method for producing high resolution computed tomography (CT) images is disclosed. The method comprises combining multiple focal spots in a single data acquisition to provide high-resolution multi-resolution data that is able to resolve fine features and with fluence to reduce noise levels, wherein the multiple focal spots have different focal spot sizes; and processing the high-resolution multi-resolution data using a model-based iterative reconstruction (MBIR) method. The method can further comprise providing the multiple focal spots using an x-ray source. The x-ray source can comprise a dual-source, dual detector CT scanner, a single-source CT scanner with different tube settings, and x-ray tubes with adjustable focal spots. The multiple focal spots comprise a first focal spot that is larger and produced with a higher power x-ray source and a second focal spot that is smaller and produced with a lower power x-ray source than the first focal spot.

According to examples of the present disclosure, a method for producing high resolution computed tomography (CT) images is disclosed. The method comprises providing a first focal spot and a second focal spot to a target location on a patient, wherein the first focal spot is larger and produced with a first x-ray source at a higher power and the second focal spot that is smaller and produced with a second x-ray source at a lower power than the first focal spot; recording one or more images from the first focal spot and recording one or more images from the second focal spot; combining the one or more images from the first focal spot the one or more images from the second focal spot to provide high-resolution multi-resolution data that is able to resolve fine features and with fluence to reduce noise levels; and processing the high-resolution multi-resolution data using a model-based iterative reconstruction (MBIR) method. The first x-ray source and the second x-ray source are the same x-ray source or different x-ray sources.

According to examples of the present disclosure, a system for producing high resolution computed tomography (CT) images is disclosed. The method comprises a first x-ray source that provides a first focal spot and a second x-ray source that provides second focal spot to a target location on a patient, wherein the first focal spot is larger and produced with the first x-ray source at a higher power and the second focal spot that is smaller and produced with the second x-ray source at a lower power than the first focal spot; a detector that detects and records one or more images from the first focal spot and recording one or more images from the second focal spot; and a computer system comprising a processor and computer-readable storable medium that stores instructions to combine the one or more images from the first focal spot the one or more images from the second focal spot to provide high-resolution multi-resolution data that is able to resolve fine features and with fluence to reduce noise levels and processes the high-resolution multi-resolution data using a model-based iterative reconstruction (MBIR) method. The first x-ray source and the second x-ray source are the same x-ray source or different x-ray sources.

### BRIEF DESCRIPTION OF THE FIGURES

FIG. 1 shows a UHR-CT workflow: CT acquisition involves collection of two sets of projection data with different focal spot settings. In currently available CT scanners this can be a large and small focal spot, but future systems can have more general focal spot designs. The multi-resolution projection data is jointly processed leveraging the increased fluence in larger spots for reduced noise and higher-resolution data (albeit with reduced fluence) in smaller focal spot data.

FIG. 2A shows an extended focal spot model that uses many sourcelets that represent the spatial distribution of x-ray emissions coming from the anode according to examples of the present disclosure.

FIG. 2B shows example strategies that use two focal spots in which one, or both, focal spots are intentionally chosen with larger areas to permit higher fluence production, but also with one small focal spot or a focal spot with high spatial frequencies in its distribution to capture high-resolution features in projection data according to examples of the present disclosure.

FIG. 3A and FIG. 3B show noise-resolution plots for each of the four protocols using the FWHM of the point stimulus as shown in FIG. 3A and relative modulation of the 20 cycles/cm sinusoidal feature as the resolution metric as shown in FIG. 3B according to examples of the present disclosure.

FIG. 4 shows sample results of simulation studies according to examples of the present disclosure: on the left, ground truth phantom with varied sinusoidal features from 13-20 cycles/cm; on the right, reconstruction results for noise-matched and resolution matched, according to the 20 cycle/cm modulation criterion, for three of the protocols.

FIG. 5A, FIG. 5B, FIG. 5C, and FIG. 5D show sample focal spot strategies for focal spot #1 (top row) and focal spot #2 (bottom row) according to examples of the present disclosure, where FIG. 5A shows a large focal spot for focal spot #1 and a small focal spot for focal spot #2, FIG. 5B shows a vertical focal spot for focal spot #1 and a horizontal focal spot for focal spot #2, FIG. 5C shows vertical line pairs for focal spot #1 and horizontal line pairs for focal spot #2, and FIG. 5D shows other structures for focal spot #1 and for focal spot #2.

FIG. 6A shows a plot of focal spots (orthogonal view on anode) according to examples of the present disclosure.

FIG. 6B shows plots of apparent focal spots versus detector position according to examples of the present disclosure, where the change in the apparent focal spot is measured using pinhole imaging.

FIG. 6C shows a focal spot model according to examples of the present disclosure, where an arbitrary intensity distribution on the anode is modeled with individual ray projections between each source-let and the detector.

FIG. 7A shows a first example of a machine learning computer model according to examples of the present disclosure.

FIG. 7B shows a second example of a machine learning computer model according to examples of the present disclosure.

FIG. 7C shows a third example of a machine learning computer model according to examples of the present disclosure.

FIG. 8 shows results from simulation studies on a plurality of digital phantoms according to examples of the present disclosure.

FIG. 9 shows a method for producing high resolution computed tomography (CT) images, according to examples of the present disclosure.

FIG. 10 show a method for producing high resolution computed tomography (CT) images according to examples of the present disclosure.

FIG. 11 shows a computing system according to examples of the present disclosure.

### DETAILED DESCRIPTION

Generally speaking, examples of the present disclosure provide for the combination of multiple focal spots in a single data acquisition to provide both the high-resolution data required to resolve fine features but also the fluence required to reduce noise levels. The disclosed techniques can use a protocol with two different focal spot sizes to produce multi-resolution data that is jointly processed using a model-based iterative reconstruction (MBIR) method. The acquisition and reconstruction scheme are compared with alternate acquisition and processing method (including large focal spot only, and small focal spot only protocols). The multiple focal spot technique exhibits superior performance over a wide range of MBIR regularization strengths—demonstrating the potential of the multi-focal spot approach to surpass traditional high-resolution performance limits.

For example, a system and method for CT data collection is provided, wherein multiple focal spots are used. Specifically, while a small focal spot can provide high-resolution, it does so with lower power limits, resulting in noisier data. The data collection can be augmented with additional projections using a larger focal spot (with higher power limitations) to reduce the overall noise in the reconstruction. A model-based reconstruction scheme is used to jointly process the higher-noise, finer-resolution projections along with the lower-noise, coarser-resolution data in an attempt to get low noise, high-resolution volumes.

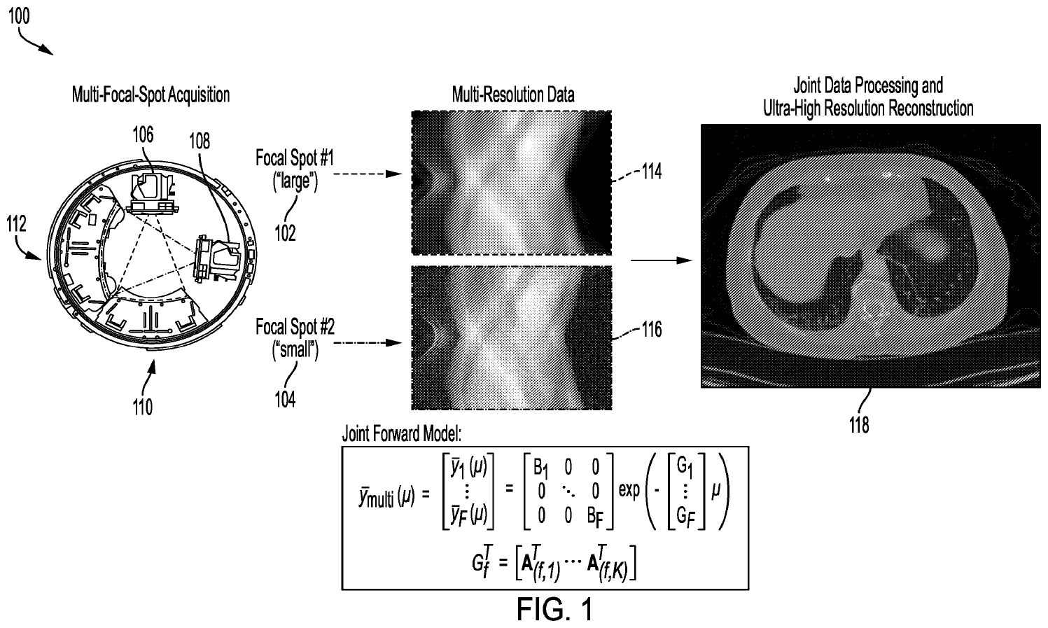

FIG. 1 shows a UHR-CT workflow 100 according to examples of the present disclosure. CT acquisition involves collection of two sets of projection data with different focal spot settings (focal spot #1 102 and focal spot #2 104) that are emitted by respective emitters 106 and 108 and collected by respective detectors 110 and 112. Although the emitters shown in FIG. 1 are orthogonally arranged, this is just one non-limiting example. Other angled configuration of emitters can be used as desired. Emitter 106 and Emitter 108 are configured to produced different types of focal spots. Emitter 106 can be configured to produce a first type of focal spot and emitter 108 can be configured to produce a second type of focal spot. The first type of focal spot and the second type of focal spot can differ in size, geometries, number of spots, or combinations thereof. In some examples, a single emitter can be used to produce the different focal spot types. In some examples, more than two emitters can be used to produce more than two types of focal spots. In some currently available CT scanners, the emitters can produce different types of focal spots, such as a large focal spot and a small focal spot, but others CT systems can have more general focal spot designs. Multi-resolution projection data 114, 116 produced by focal spot #1 102 and focal spot #2 104, respectively, are jointly processed into a UHR-CT image 118 leveraging the increased fluence in larger spots for reduced noise and higher-resolution data (albeit with reduced fluence) in smaller focal spot data.

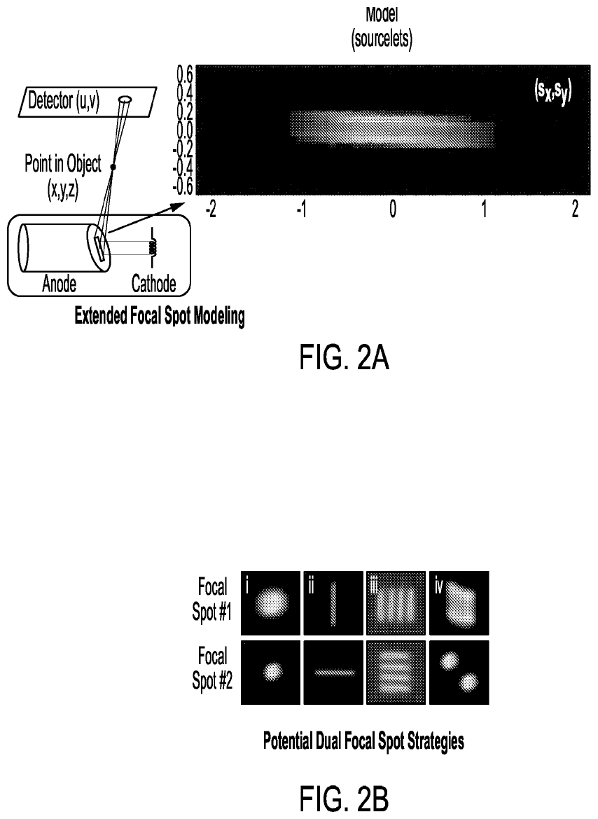

FIG. 2A shows an extended focal spot model that uses many sourcelets that represent the spatial distribution of x-ray emissions coming from the anode according to examples of the present disclosure and FIG. 2B shows example strategies that use two focal spots in which one, or both, focal spots are intentionally chosen with larger areas to permit higher fluence production, but also with one small focal spot or a focal spot with high spatial frequencies in its distribution to capture high-resolution features in projection data according to examples of the present disclosure.

As shown in FIG. 2A, blur induced by an extended x-ray focal spot can be complex. A data acquisition model with source blur effects is illustrated in FIG. 2A. It common for x-ray tubes to focus the electron beam emitted from cathode 202 to strike a target of anode 204 with a pattern that is elongated and roughly line-like. With anode 204 with a shallow angle, this results in an apparent focal spot that is more point-like due to its oblique viewing angle with respect to detector 206. The larger area on anode 204 permits greater heat and power dissipation. However, even at shallow angles, the source can appear extended and features within the object will be blurred. This can be seen by breaking the extended source into many “sourcelets” 208—each of which can form a projection on detector 206. The induced blur varies with the position in the object: e.g, increased blur for points closer to the source; and varying apparent focal spot size based on the angle between the object point and the source.

The system models and reconstruction for data acquisition with adaptive focal spots is now discussed. The high-fidelity models for photon-counting CT can include source blur. The following general model is used where the mean measurements are represented as

$$\overline{y} = B exp ( - A μ )$$

where μ denotes the volume of attenuation coefficients representing the patient, A is the so-called system matrix that performs the linear projection operation and B is another matrix operation that can model multiple effects. In a standard, simple forward model, B is a scalar or a diagonal matrix that represents the mean bare-beam fluence (possibly varying across measurements). However, the general form can accommodate much more sophisticated physical models. For example, B can represent a detector blur (a linear operation that may be applied with a convolution). In this disclosure, a high-fidelity model of source blur may be formed in the following fashion. Rather than letting elements of A, aᵢⱼ, denote the contributions of the j^{th} voxel to the i^{th} measurement along a line connecting the x-ray source and the pixel associated with i, the source can be subsampled into many “sourcelets.” This results in many more rays that connect the source and each detector pixel, which may be weighted (e.g. by the sourcelet intensity) and summed (post-exponentiation) using B. This model may similarly model exponential edge-gradient effects using “sub-pixels” which are also integrated over the pixel aperture.

Focal spot blur can be mitigated by explicitly modeling the extended source within a model-based iterative reconstruction (MBIR) technique. The forward model and optimization for that approach are as follows. The forward model for a single focal spot with K sourcelets (to model the blur associated with that focal spot) can be expressed as follows:

$${\overline{y}}_{multi} ( μ ) = [ \begin{matrix}\begin{matrix}{\overline{y}}_{1} ( μ ) \\ ⋮\end{matrix} \\ {\overline{y}}_{F} ( μ )\end{matrix} ] = [ \begin{matrix}{B}_{1} & 0 & 0 \\ 0 & ⋱ & 0 \\ 0 & 0 & {B}_{F}\end{matrix} ] exp ( - [ \begin{matrix}\begin{matrix}{G}_{1} \\ ⋮\end{matrix} \\ {G}_{F}\end{matrix} ] μ )$$

A forward model for multiple focal spots (each G_{f} uses many sourcelets to model blur) can be expressed as follows:

$$\hat{μ} = arg {{min}_{μ} [ y - {\overline{y}}_{m u l t i} ( μ ) ]}^{T} W [ y - {\overline{y}}_{m u l t i} ( μ ) ] + β R ( μ )$$

$${G}_{f}^{T} = [ {A}_{( f , 1 )}^{T} … {A}_{( f , K )}^{T} ]$$

where y̅_{f} denotes measurements associated with the f^{th} focal spot, G_{f} is the collection of system matrices for all sourcelets in the f^{th} focal spot, and B_{f} sums over all sourcelets for the f^{th} focal spot. It is noted that the same nonlinear least-squares objective may be used for reconstruction. Moreover, since the forward model fits the same form as Bexp(Aμ), the same iterative algorithm as posed in Tilley et al. can be used, as described above in paragraph [0030] where a forward model that is a linear operator, an exponential, and then a linear operator is used. In short, the model uses optimization transfer, where the objection function described in paragraph [0032] is successively approximated by so-called surrogate functions which are minimized to yield a new estimate. New surrogates are created at this estimate, which are then minimized. The process continues iteratively. Other optimization approaches, e.g., gradient methods, stochastic optimization could also be used.

Model-based reconstruction objective for multi-focal spot processing can be expressed as follows:

$$\overline{y} ( u ) = B exp ( - G μ ) = [ {s}_{1} I … {s}_{K} I ] exp ( \begin{matrix}\begin{matrix}{A}_{1} \\ ⋮\end{matrix} \\ {A}_{K}\end{matrix} )$$

The MBIR estimate \hat{μ} is found using a weighted least-squares difference between the projection data y and the forward model y̅(μ)weighted by the inverse of the variance of measurements W and a regularization term βR(μ). The regularization term can be any number of equations including classic quadradic penalties on pairwise neighboring voxel differences, other more general Markov random field priors with nonquadratic terms, etc.

This MBIR method permits higher spatial resolution reconstructions, but does so at the cost of increased noise. In some examples, an alternate strategy is provided where both high- and low-spatial resolution data are collected to obtain high resolution and relatively low noise reconstruction. There are various ways to accomplish such an acquisition including through currently available dual-source, dual detector CT scanners; multiple acquisitions with different tube settings; and tube designs with adjustable focal spots. One can envision many different focal spot designs that balance the maximum fluence and spatial resolution (see FIG. 2B). It will be concentrated here on the case of a small focal spot operating at “maximum” power that is augmented by a larger focal spot for noise reduction, as in FIG. 2B.

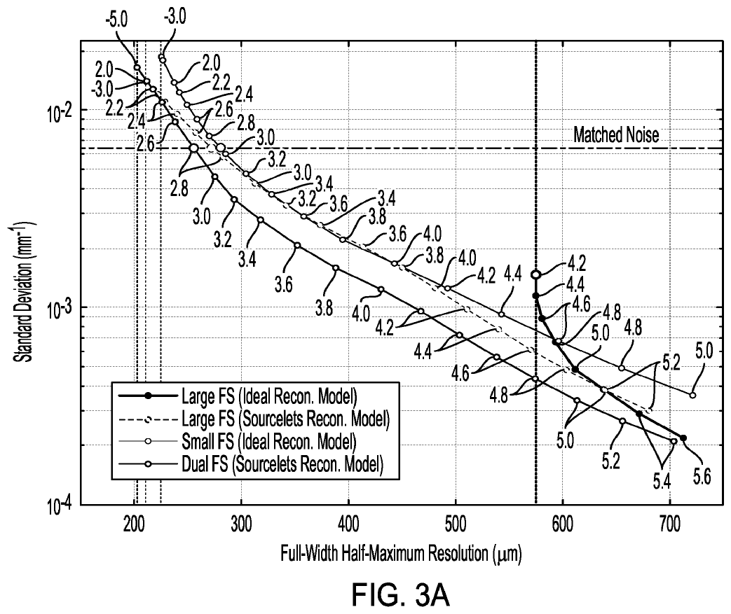

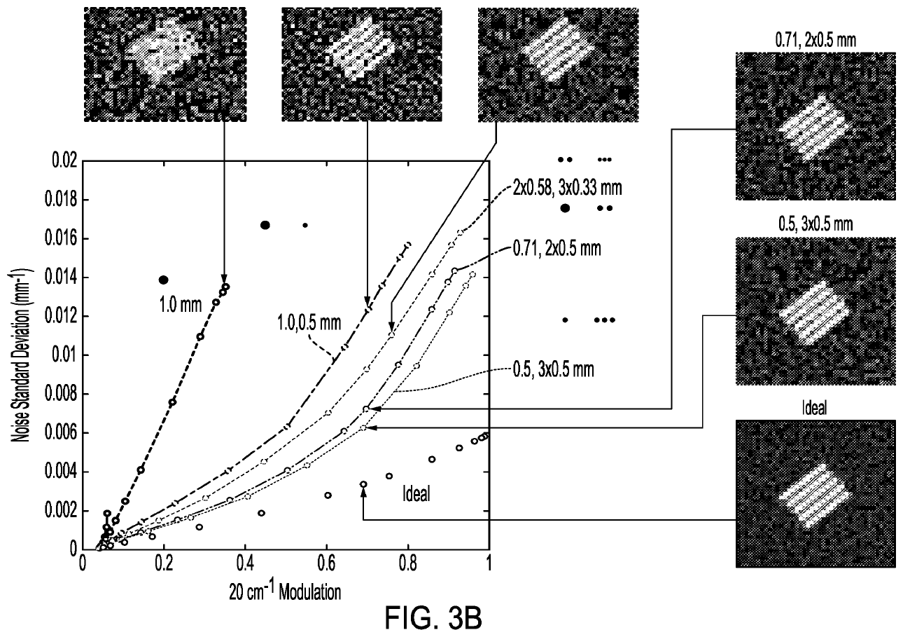

FIG. 3A and FIG. 3B show noise-resolution plots for each of the four protocols using the FWHM of the point stimulus as shown in FIG. 3A and relative modulation of the 20 cycles/cm sinusoidal feature as the resolution metric as shown in FIG. 3B. Limiting spatial resolution (where lower R do not improve the resolution metric) is identified by vertical dotted lines. Matched noise and matched resolution scenarios are also identified.

To investigate the dual focal spot approach and compare the relative performance of difference protocols, a set of simulation studies was conducted. Four different acquisition and reconstruction protocols were studied: The first was for a large focal spot with ideal model −5×1 mm focal spot (on the surface of the anode) at a 10° angle; 104 photons/pixel barebeam; reconstruction model with a single ideal point source. The second was for a large focal spot with sourcelet focal spot model −5×1 mm focal spot at a 10° angle; 104 photons/pixel; reconstruction model with a 5×5 array of sourcelets and uniform intensity distribution. The third was for a small focal spot with ideal model −1×0.4 mm focal spot at a 10° angle; 741 photons/pixel (scaled to same fluence/anode area as large focal spot); reconstruction model with a single ideal point source. The fourth was for a dual focal spots with sourcelet model −5×1 mm and a 1×0.4 mm focal spot, both at a 10° angle; 9259 and 741 photons/pixel, respectively (for a total of 104); reconstruction model with a 5×5 array of sourcelets and uniform intensity distribution for each source.

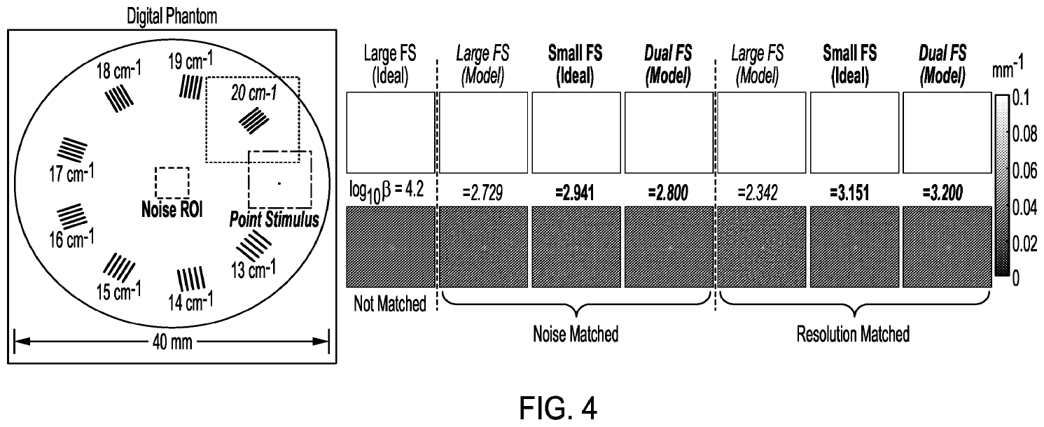

All studies used a system geometry including: 120 cm source-to-detector, 60 cm source-to-axis, 360 projection angles, a 400×45 detector with 0.25 mm square pixels. A cylindrical digital phantom (40 mm diameter, see FIG. 4) with uniform attenuation, sinusoidal features from 13 to 20 cycles/cm, and a single point stimulus was used for studies. Reconstruction used 100 iterations of the algorithm presented in Tilley et al. with 10 subsets, 0.2 mm voxels, Nesterov acceleration, a separable footprint projector, and a quadratic roughness penalty on first order voxel differences. To evaluate the performance of each method, a series of reconstructions was performed varying the regularization parameter β logarithmically. Noise resolution plots were generated two ways. Sample standard deviation was computed in a central uniform region to estimate noise. Resolution was computed two ways: 1) the full-width, half-maximum of the reconstructed point source; and 2) by find the relative degree of modulation of the 20 cycles/cm sinusoidal features. Resolution metrics were computed using noiseless data.

FIG. 4 shows sample results of the simulation studies: on the left, ground truth phantom with varied sinusoidal features from 13-20 cycles/cm; on the right, reconstruction results for noise-matched and resolution matched, according to the 20 cycle/cm modulation criterion, for three of the protocols. Note that the large focal spot (ideal) protocol could not be matched but a sample unmatched reconstruction is shown.

During investigations it is noted that the shape of the point stimulus in reconstruction was varied (e.g. in some cases with noticeable sidelobes, or a flat-topped peak)—making comparisons difficult. In contrast more consistent results were observed with the relative modulation metric. The noise-resolution curves using each metric and for each protocol are presented in FIG. 2A and FIG. 2B. The following trends were observed. The large focal spot with an ideal model is resolution-limited (at around 575 μm). Including a sourcelet model extends the spatial resolution range but at the cost of increased noise. The small focal spot acquisition can achieve high spatial resolution but at higher noise levels. Interestingly, the performance is similar to the large focal spot acquisition with sourcelet model over a range of spatial resolution with the FWHM metric, but it is consistently better for the 20 cycle/cm modulation metric. The dual focal spot consistently performs best across the entire range of spatial resolutions with the lowest noise reconstructions. (It is noted there is an apparent similarity in performance of the large focal spot with sourcelet model case for very high spatial resolutions, but this is also where the large focal spot case has an unusual reconstruction of the point stimulus.) To further illustrate performance differences matched noise and matched resolution reconstruction (based on the 20 cycles/cm metric) are shown in FIG. 3A and FIG. 3B. (Unmatched large focal spot reconstructions are presented since this protocol cannot achieve the prescribed performance.) The observations from the noise-resolution curves are evident in the reconstructions. For example, at matched noise, the sinusoidal modulation is best for the dual focal spot scenario and worst for the large focal spot/sourcelet model (not including the large, ideal case). Similarly, at matched spatial resolution the dual focal spot protocol has the lowest noise, whereas the large focal spot/sourcelet model case is most noisy.

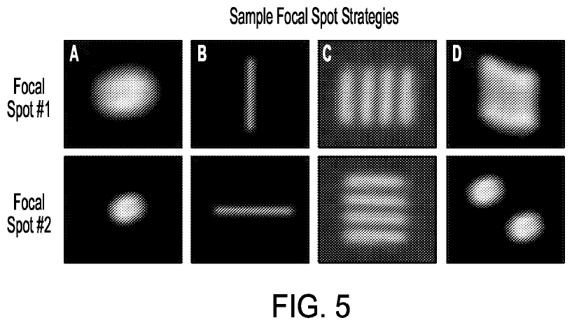

FIG. 5A, FIG. 5B, FIG. 5C, and FIG. 5D show sample focal spot strategies for focal spot #1 (top row) and focal spot #2 (bottom row), where FIG. 5A shows a large focal spot for focal spot #1 and a small focal spot for focal spot #2, FIG. 5B shows a vertical focal spot for focal spot #1 and a horizontal focal spot for focal spot #2, FIG. 5C shows vertical line pairs for focal spot #1 and horizontal line pairs for focal spot #2, and FIG. 5D shows other structures for focal spot #1 and for focal spot #2.

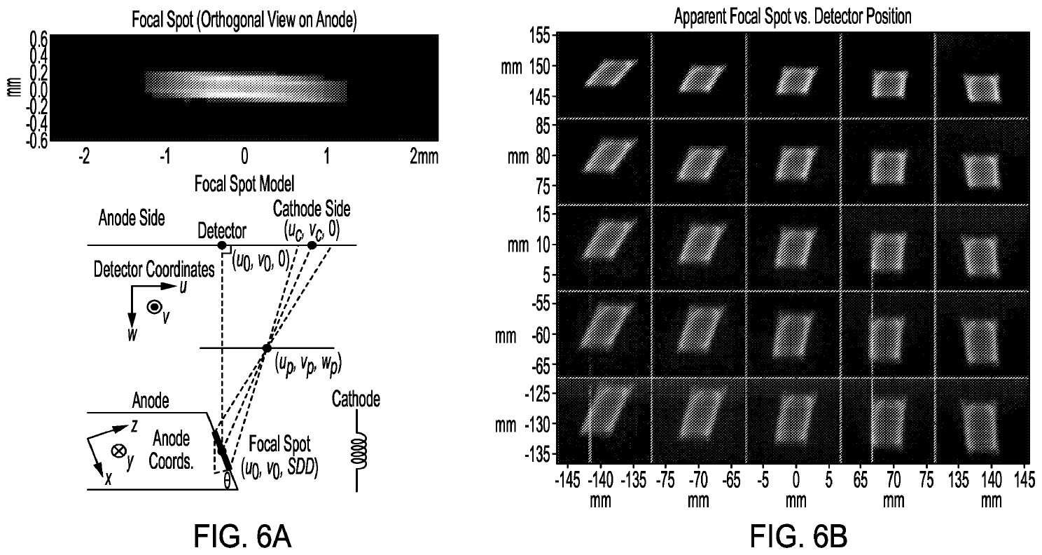

FIG. 6A shows a plot of focal spots (orthogonal view on anode) according to examples of the present disclosure. FIG. 6B shows plots of apparent focal spots versus detector position according to examples of the present disclosure, where the change in the apparent focal spot is measured using pinhole imaging. FIG. 6C shows a focal spot model according to examples of the present disclosure, where an arbitrary intensity distribution on the anode is modeled with individual ray projections between each source-let and the detector. This model captures the depth-dependent and shift-variant nature of focal spot blur.

This mathematical model can be used to represent a CT system with shift-variant and depth-dependent source blur. Data illustrating the application of the sourcelet model is shown in FIG. 5B where an x-ray tube with an obliquely angled anode and an elongated (i.e., line source), nonuniform focal spot is modeled. Both the depth-dependence of source blur (more blur closer to the focal spot) and the shift-variant nature (changing apparent focal spot size as a function of detector position) are accommodated. For the disclosed methods and systems, A and B can be similarly expanded to encompass different combinations of focal spots for different sets of measurements—e.g., projections with both large and small focal spots, or focal spots with structured shape, etc.

The above model for photon-counting CT systems can be extended. Refinements can include adaptations to currently available photon counters (e.g. pixel-size, geometry, sensitivity, etc.), common gantry geometries, as well as arbitrary focal spot distributions and structured focal spots (like those shown in FIG. 5A, FIG. 5B, FIG. 5C, and FIG. 5D). Because the high-resolution application inherently requires smaller pixels and smaller voxels (even without pixel or sourcelet subsampling), the computationally efficient methods are used that maintain physically accurate system models. For example, a separable sourcelet projector can be implemented on GPU for fast computation.

The high-fidelity model can be inverted using model-based approaches. The following general least-squares objective function is adapted to be minimized:

$$ψ \frac{1}{2} {( y - {Be}^{- A μ} )}^{T} W ( y - B {e}^{- A μ} ) + β R ( μ ) ,$$

where noisy measurements are denoted by y, and W denotes the inverse of the covariance matrix associated with the data. A regularization term, R(μ) with strength β, allows for general incorporation of prior knowledge. (FIG. 4 preliminary data used a simple pairwise quadratic penalty.) An algorithm, as described above in paragraph [0032] with, is used to solve this objective iteratively for arbitrary A, B, and W. This objective is applied in data with noise correlations, projections with shift-variant focal spot blur, and multi-resolution dual-layer detector flat-panel CT. The same underlying methodology can be applied to the UHR-CT technique with adaptive focal spots. Translation to UHR-CT requires the integration of the high-fidelity models. Some modifications (e.g. additive scatter in the model) may require changes to the above objective, but are unlikely to require a re-derivation of the underlying algorithm. A quadratic regularization can be used as a baseline approach and make comparisons to edge-preserving non-quadratic penalties that are likely to improve high-spatial resolution performance. More sophisticated techniques based on deep learning using MBIR with a machine-learned trained modeled using an encoder-decoder network can also be used. Other algorithmic analysis can be used including convergence studies (rate, monotonicity, local optima, etc.) and the effect of different algorithmic strategies (e.g. common speed-ups like ordered-subsets, Nesterov acceleration based on momentum, regularization, etc.). Such analysis can be performed to ensure reliable reconstruction, establish protocols (including number of iterations or stopping criteria).

According to examples of the present disclosure, a deep-learning or machine-learning neural network reconstruction can be constructed and used to jointly process multi-resolution CT data While MBIR methods permit direct integration of physical models into the image formation process, such iterative algorithms are often computationally expensive. With decreasing pixel and voxel size, and increasingly sophisticated forward models, such burdens are likely to increase. In parallel with the MBIR development, data-driven deep-learning approaches can be used with their ability to integrate generalized prior information and their relative computational speed. Three different classes of processing architectures are described below.

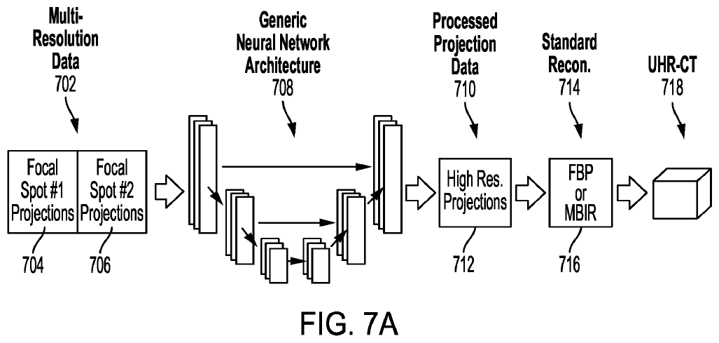

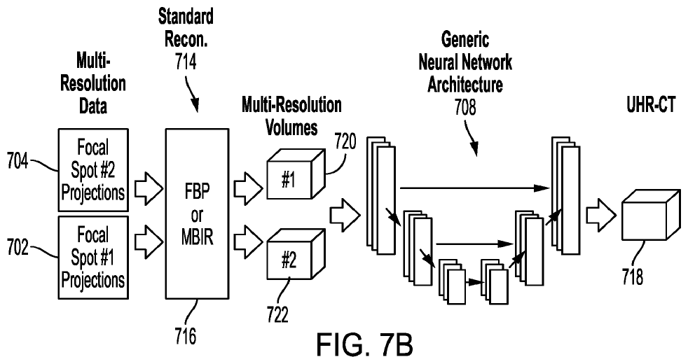

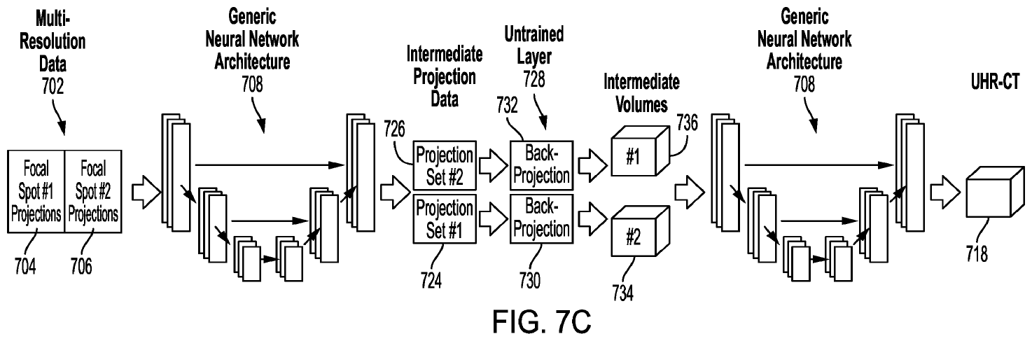

FIG. 7A shows a first example of a machine learning computer model according to examples of the present disclosure. This first example is termed Project-Domain Processing (PDP). When two focal spots are used to obtain multiresolution projection data where every projection view was acquired with each focal spot, there is an opportunity to perform a kind of multi-frame restoration in the projection-domain. In this model, a projection pair is given as an input to a neural network that seeks a high-resolution (restored) projection. All pre-processed projections can then be reconstructed using standard methods into a UHR-CT volume. In this model, projection pairs inputs and high-resolution projection “labels” are used for network training. Such an approach is an extension of work performed by Kuntz et al but with multi-resolution inputs. As shown in FIG. 7A, multi-resolution data 702 in the form of focal spot #1 projections 704 and focal spot #2 projections 706, are collectively provided to generic neural network architecture 708 that collectively processes multi-resolution data 702 to produce processed projection data 710 in the form of high-resolution projections 712. Processed projection data 710 is then further processed using standard reconstruction techniques 714 to yield filtered backprojection (FBP) or model-based iterative reconstruction (MBIR) 716. Finally, ultra-high resolution CT image(s) 718 are produced.

FIG. 7B shows a second example of a machine learning computer model according to examples of the present disclosure. This second example is termed Image-Domain Processing (IDP). An alternative processing approach is to first reconstruct each set of focal spot data into its own volume. Akin to image-domain processing of spectral data, the original projection datasets do not need to be coincident (as with projection-domain processing). Following reconstruction, two volumes with differing spatial resolutions are available for input to a neural network responsible for combining them into a single UHR-CT volume. This approach has similarities with multiresolution image fusion that has been used in various imaging modalities. Here training uses paired reconstructed volumes for each focal spot as inputs and a high-resolution UHR-CT volume is the “label”. As shown in FIG. 7B, multi-resolution data 702 in the form of focal spot #1 projections 704 and focal spot #2 projections 706, are individually provided to standard reconstruction techniques 714 to yield FBP or model-based iterative reconstruction (MBIR) 716 that individually processes multi-resolution data 702 to produce multi-resolution volume #1 720 and multi-resolution volume #2 722, which are then provided to and processed by generic neural network architecture 708 that produces ultra-high resolution CT image(s) 718.

FIG. 7C shows a third example of a machine learning computer model according to examples of the present disclosure. This third example is termed End-to-end Processing (EEP). A third option uses both neural network processing of projection data as well as image-domain processing. This approach is enabled by a central untrained layer that performs backprojection on intermediate/processed projection data. End-to-end training has the potential to leverage both projection- and image-domain computations for improved performance as well as the incorporation of known physical relationships (e.g. known geometric aspects, pixel sampling, etc.) in the untrained layer. For this processing, training pairs include multiresolution projection inputs and the UHR-CT ground truth volume as “labels.” As shown in FIG. 7C, multi-resolution data 702 in the form of focal spot #1 projections 704 and focal spot #2 projections 706, are collectively provided to and processed by a first generic neural network architecture, such as generic neural network 708, that collectively processes multi-resolution data 702 to produce intermediate projection data set #1 724 and intermediate projection set #2 726. Untrained layer 728 performs back-projection operations 730 and 732 on intermediate projection data set #1 724 and intermediate projection set #2 726, respectively, which then yields intermediate volume #1 734 and intermediate volume #2 736. Intermediate volume #1 734 and intermediate volume #2 736 are then provided to a second generic neural network architecture, such as generic neural network 708, to produce ultra-high resolution CT image(s) 718.

The above-disclosed machine learning computer models can be configured using one or more sources of data including the following: 1) Procedurally generated phantoms (as used in Russ et al.⁴⁴ and Shi et al.⁴⁵) permits arbitrarily high-resolution simulations and large datasets, though with limited anthropomorphic realism. 2) The XCAT phantom and anthropomorphic digital phantom with realistic anatomy and arbitrarily high resolution (though limited in texture). This phantom was previously used for neural network training in related deep-learning reconstruction development. 3) Online databases including TCIA and LIDC. These are public anonymized patient studies that have used previously in neural network training for lung nodule synthesis. They are anatomically realistic but potentially limited in spatial resolution. As appropriate, modifications to these dataset can be considered—e.g. with the focus on thoracic and pulmonary imaging where additional high-resolution lung textures can be injected into realistic LIDC data, etc. to accommodate the joint goals of high-resolution training data but also realistic physical properties/anatomy. Regardless of the digital phantom source, all data will use the Aim 1.1 model to generate realistic projections for subsequent training.

The above methods are illustrated with an arbitrary network structure using one or more GPUs or similar processing architectures. Different network architectures can be used as they are used in similar applications. For example, convolutional neural networks have found success in other deblurring applications and can be used for PDP, GANs have been used for multiresolution fusion and IDP, etc. Common neural network validation tests can be applied and include generalization studies (looking at data outside the training set), ablation studies (to consider network efficiency), etc.

In some examples, many different UHR-CT strategies can be used. An adaptive focal spot strategy for UHR-CT can be used that seeks to balance high-resolution information (e.g. via small focal spots) with coarser data that has higher fluence and the ability to reduce noise. In essence, this is balancing focal spots distributions that can obtain high-frequency information and those that deliver high fluence. This can include many designs such as those illustrated in FIG. 5A that shows small and large focal spots (as available on current systems), FIG. 5B that shows horizontal and vertical line sources, FIG. 5C that shows multiple line structures (e.g. repeated bar patterns at different orientations), and FIG. 5D that shows multiple isolated small focal spots (with regular or irregular positioning). Although not shown, various combinations of these methods can be used.

A focal spot model can be used that that governs the maximum fluence that can be generated for a particular design is that the maximum current is proportional to the area of the focal spot. Other physical effects including the limitations of electron beam optics and off-focal radiation can be included in the model. When possible (e.g. with standard round or square focal spots), simulation can use focal spot distributions based on pinhole measurements. Realistic focal spot distributions for designs can be designed through selective modeling (e.g. of electron optics) via Monte Carlo simulations. A simulation in can be set up as follows: X-ray generation can be simulated in a vacuum. Electrons are launched directly from the cathode at the beginning of the process. These electrons are launched with predetermined moment vectors and kinetic energies. The anode can be constructed with tungsten as the target material and can be inclined at 8 degrees (similar to the tube design in the PCCT system). The electron emission can be simulated using predefined modeling functions as a circular focal point with an adjustable radius. The incoming energy to the anode can be modeled as a Gaussian distribution for a round focal spot. As part of the simulation of the focal spot designs, multiple circular focal points can be combined to form one or more desired geometries. In addition to the model of focal spot distributions, other acquisition protocols can be modeled. In particular, the particular balance of fluence between focal spots can be modeled. Similarly, the impact of changing the number of projections acquired using each focal spot can be modeled. It is noted that most modern CT scanners employ tube current modulation and bow-tie filters. Both classic current modulation techniques and a reasonably sized bow-tie filter appropriate for thoracic/pulmonary imaging can be modeled. Protocols with equivalent exposures for fair comparisons can also be modeled.

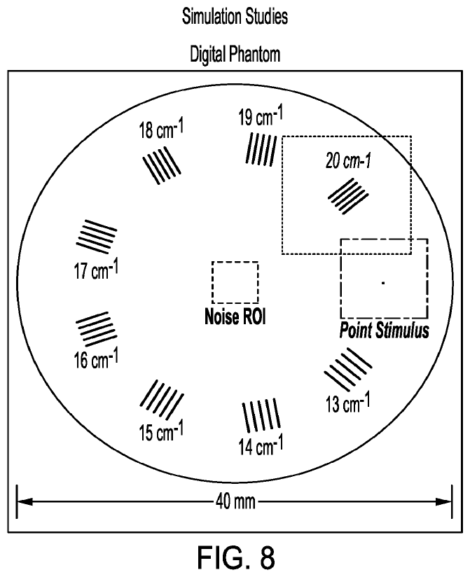

FIG. 8 shows results from simulation studies on a plurality of digital phantoms according to examples of the present disclosure.

In some examples, the simulation studies can be performed using a platform including a post-processing workstation and an offline reconstruction engine for dedicated computations (outside of the clinically workflow). In addition, the platform can provide access to preprocessed projection image data for custom processing and reconstruction. For the purpose of reconstruction and modeling tasks, preliminary data revealed that signal statistics are governed by a compound Poisson distribution, and electronic background noise is removed at ultra-low dose acquisitions, and provides for precise and reliable HUs at ultra-low dose levels.

An example data collection and pre-processing pipeline that can used is as follows: (i) detector configuration is in a high-resolution mode (0.2 mm slice thickness) using all photons above the lowest energy threshold (approx. 25 keV/allowing to remove electronic background noise), (ii) data is acquired with a tube voltage of 120 kVp and pitch of 0.5 with all dose modulation functions switched off, (iii) in order to access the different focal spot sizes (0.4×0.5 mm, 0.6×0.7 mm, 0.8×1.1 mm), the tube current is adapted, resulting in dose values between 0.4 mGy and 20 mGy, (iv) slices are reconstructed with a dedicated vendor-specific reconstruction workstation with matrix sizes 512×512, 1024×1024, and 2048×2048 with dedicated high resolution kernels, and (v) projection image data and reconstructed slices are transferred to a PACS system.

In summary, a framework for high-spatial-resolution CT acquisition and reconstruction is provided, wherein multiple x-ray focal spots (of varying size/structure) are used to produce multi-resolution data. Through joint processing of the data, high-resolution features can be extracted from a small but fluence-limited dataset and the overall noise can be reduced through the larger, higher fluence data. This technology offers a strategy to improve high-spatial resolution CT using currently available systems—e.g. dual-source scanners with different sized x-ray focal spots; but also to enable the design of new CT systems with new focal spot designs.

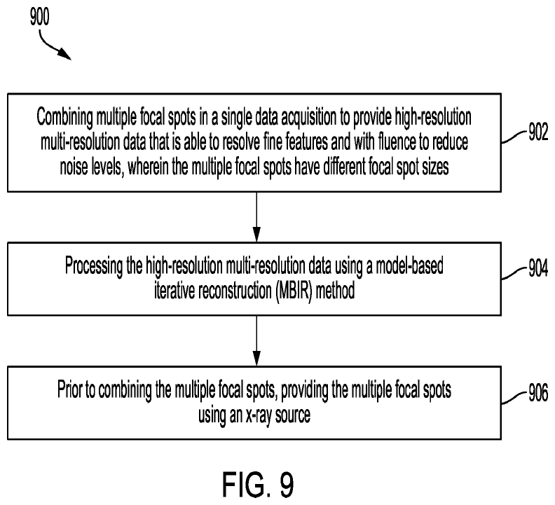

FIG. 9 shows method 900 for producing high resolution computed tomography (CT) images, according to examples of the present disclosure. Method 900 comprises combining multiple focal spots in a single data acquisition to provide high-resolution multi-resolution data that is able to resolve fine features and with fluence to reduce noise levels, wherein the multiple focal spots have different focal spot sizes, as in 902. Method 900 continues by processing the high-resolution multi-resolution data using a model-based iterative reconstruction (MBIR) method, as in 904. Method 900 can comprise, prior to combining the multiple focal spots, method 900 can comprise providing the multiple focal spots using an x-ray source, as in 906. The x-ray source can comprise a dual-source, dual detector CT scanner, a single-source CT scanner with different tube settings, and x-ray tubes with adjustable focal spots. The multiple focal spots can comprise a first focal spot that is larger and produced with a higher power x-ray source and a second focal spot that is smaller and produced with a lower power x-ray source than the first focal spot.

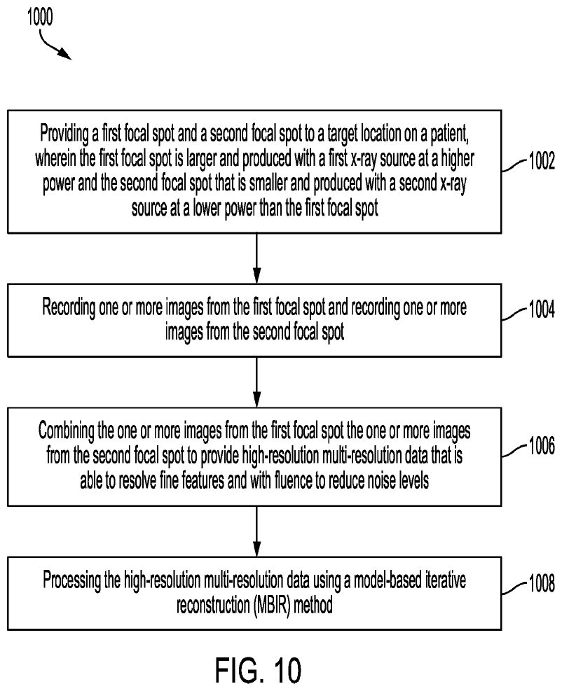

FIG. 10 show method 1000 for producing high resolution computed tomography (CT) images according to examples of the present disclosure. Method 1000 comprises providing a first focal spot and a second focal spot to a target location on a patient, wherein the first focal spot is larger and produced with a first x-ray source at a higher power and the second focal spot that is smaller and produced with a second x-ray source at a lower power than the first focal spot, as in 1002. Method 1000 continues by recording one or more images from the first focal spot and recording one or more images from the second focal spot, as on 1004. Method 1000 continues by combining the one or more images from the first focal spot the one or more images from the second focal spot to provide high-resolution multi-resolution data that is able to resolve fine features and with fluence to reduce noise levels, as in 1006. Method 1000 continues by processing the high-resolution multi-resolution data using a model-based iterative reconstruction (MBIR) method, as in 1008. The first x-ray source and the second x-ray source are the same x-ray source or different x-ray sources.

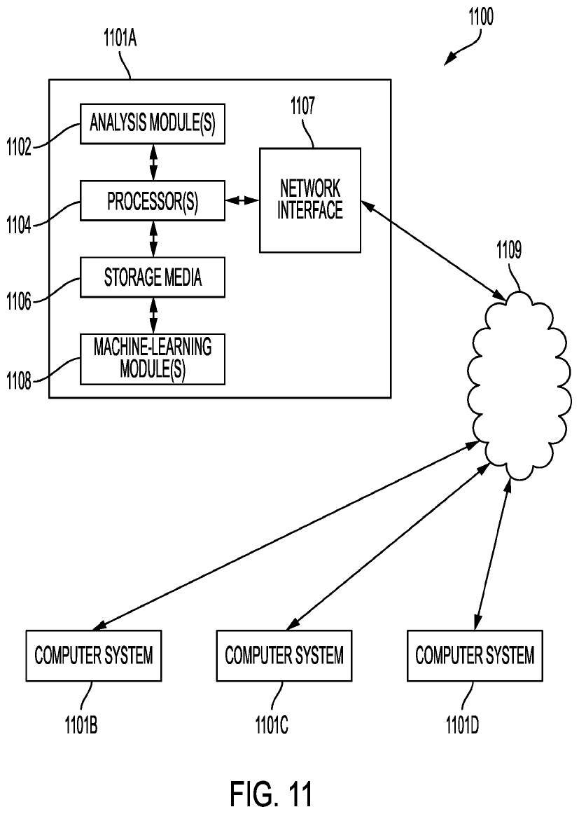

In some embodiments, any of the methods of the present disclosure may be executed by a computing system. FIG. 11 illustrates an example of such a computing system 1100, in accordance with some embodiments. The computing system 1100 may include a computer or computer system 1101A, which may be an individual computer system 1101A or an arrangement of distributed computer systems. The computer system 1101A includes one or more analysis module(s) 1102 configured to perform various tasks according to some embodiments, such as one or more methods disclosed herein. To perform these various tasks, the analysis module 1102 executes independently, or in coordination with, one or more hardware processors 1104, which is (or are) connected to one or more non-transitory computer readable medium 1106, such as a computer storage media. The hardware processor(s) 1104 is (or are) also connected to a network interface 1107 to allow the computer system 1101A to communicate over a data network 1109 with one or more additional computer systems and/or computing systems, such as 1101B, 1101C, and/or 1101D (note that computer systems 1101B, 1101C and/or 1101D may or may not share the same architecture as computer system 1101A, and may be located in different physical locations, e.g., computer systems 1101A and 1101B may be located in a processing facility, while in communication with one or more computer systems such as 1101C and/or 1101D that are located in one or more data centers, and/or located in varying countries on different continents). A processor can include a microprocessor, microcontroller, processor module or subsystem, programmable integrated circuit, programmable gate array, or another control or computing device.

The non-transitory computer readable medium 1106 can be implemented as one or more computer-readable or machine-readable storage media. The non-transitory computer readable medium 1106 can be connected to or coupled with a machine learning module(s) 1108. Note that while in the example embodiment of FIG. 11 non-transitory computer readable medium 1106 is depicted as within computer system 1101A, in some embodiments, non-transitory computer readable medium 1106 may be distributed within and/or across multiple internal and/or external enclosures of computing system 1101A and/or additional computing systems. The non-transitory computer readable medium 1106 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLURAY® disks, or other types of optical storage, or other types of storage devices. Note that the instructions discussed above can be provided on one computer-readable or machine-readable storage medium, or alternatively, can be provided on multiple computer-readable or machine-readable storage media distributed in a large system having possibly plural nodes. Such computer-readable or machine-readable storage medium or media is (are) considered to be part of an article (or article of manufacture). An article or article of manufacture can refer to any manufactured single component or multiple components. The storage medium or media can be located either in the machine running the machine-readable instructions or located at a remote site from which machine-readable instructions can be downloaded over a network for execution.

It should be appreciated that computer system 1100 (or computing system) is only one example of a computing system, and that computer system 1100 may have more or fewer components than shown, may combine additional components not depicted in the example embodiment of FIG. 11, and/or computer system 1100 may have a different configuration or arrangement of the components depicted in FIG. 11. The various components shown in FIG. 11 may be implemented in hardware, software, or a combination of both hardware and software, including one or more signal processing and/or application specific integrated circuits.

Further, the steps in the processing methods described herein may be implemented by running one or more functional modules in an information processing apparatus such as general-purpose processors or application specific chips, such as ASICs, FPGAs, PLDs, or other appropriate devices. These modules, combinations of these modules, and/or their combination with general hardware are all included within the scope of protection of the invention.

Models and/or other interpretation aids may be refined in an iterative fashion; this concept is applicable to embodiments of the present methods discussed herein. This can include use of feedback loops executed on an algorithmic basis, such as at a computing device (e.g., computing system 1100, FIG. 11), and/or through manual control by a user who may make determinations regarding whether a given step, action, template, model, or set of curves has become sufficiently accurate for the evaluation of the signal(s) under consideration.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. Moreover, the order in which the elements of the methods are illustrated and described may be re-arranged, and/or two or more elements may occur simultaneously. The embodiments were chosen and described in order to best explain the principles of the invention and its practical applications, to thereby enable others skilled in the art to best utilize the invention and various embodiments with various modifications as are suited to the particular use contemplated.

Notwithstanding that the numerical ranges and parameters setting forth the broad scope of the embodiments are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume negative values, e.g. −1, −2, −3, −10, −20, −30, etc.

The following embodiments are described for illustrative purposes only with reference to the Figures. Those of skill in the art will appreciate that the following description is exemplary in nature, and that various modifications to the parameters set forth herein could be made without departing from the scope of the present embodiments. It is intended that the specification and examples be considered as examples only. The various embodiments are not necessarily mutually exclusive, as some embodiments can be combined with one or more other embodiments to form new embodiments.

While the embodiments have been illustrated respect to one or more implementations, alterations and/or modifications can be made to the illustrated examples without departing from the spirit and scope of the appended claims. In addition, while a particular feature of the embodiments may have been disclosed with respect to only one of several implementations, such feature may be combined with one or more other features of the other implementations as may be desired and advantageous for any given or particular function.

Furthermore, to the extent that the terms “including”, “includes”, “having”, “has”, “with”, or variants thereof are used in either the detailed description and the claims, such terms are intended to be inclusive in a manner similar to the term “comprising.” As used herein, the phrase “one or more of”, for example, A, B, and C means any of the following: either A, B, or C alone; or combinations of two, such as A and B, B and C, and A and C; or combinations of A, B and C.

Other embodiments will be apparent to those skilled in the art from consideration of the specification and practice of the descriptions disclosed herein. It is intended that the specification and examples be considered as exemplary only, with a true scope and spirit of the embodiments being indicated by the following claims.

What is claimed is:

1. A method for producing high resolution computed tomography (CT) images, the method comprising:
combining multiple focal spots in a single data acquisition to provide high-resolution multi-resolution data that is able to resolve fine features and with fluence to reduce noise levels, wherein the multiple focal spots have different focal spot types; and
processing the high-resolution multi-resolution data using a model-based iterative reconstruction (MBIR) method.

2. The method of claim 1, further comprising providing the multiple focal spots using one or more x-ray sources.

3. The method of claim 2, wherein the one or more x-ray sources comprises a dual-source, dual detector CT scanner, a single-source CT scanner with different tube settings, and x-ray tubes with adjustable focal spots.

4. The method of claim 1, wherein the multiple focal spots comprise a first focal spot that is larger and produced with a higher power x-ray source and a second focal spot that is smaller and produced with a lower power x-ray source than the first focal spot.

5. The method of claim 1, wherein the different focal spot types comprise different sizes, different geometries, different number of focal spots, or combinations thereof.

6. The method of claim 1, wherein the model-based iterative reconstruction (MBIR) method uses a trained or untrained neural network.

7. The method of claim 1, wherein the model-based iterative reconstruction (MBIR) method processes the multiple focal spots sequentially.

8. The method of claim 1, wherein the model-based iterative reconstruction (MBIR) method processes the multiple focal spots concurrently.

9. A method for producing high resolution computed tomography (CT) images, the method comprising:
providing a first focal spot and a second focal spot to a target location on a patient, wherein the first focal spot is larger and produced with a first x-ray source at a higher power and the second focal spot that is smaller and produced with a second x-ray source at a lower power than the first focal spot;
recording one or more images from the first focal spot and recording one or more images from the second focal spot;
combining the one or more images from the first focal spot the one or more images from the second focal spot to provide high-resolution multi-resolution data that is able to resolve fine features and with fluence to reduce noise levels; and
processing the high-resolution multi-resolution data using a model-based iterative reconstruction (MBIR) method.

10. The method of claim 9, wherein the first x-ray source and the second x-ray source are the same x-ray source or different x-ray sources.

11. A system for producing high resolution computed tomography (CT) images, the method comprising:
a first x-ray source that provides a first focal spot and a second x-ray source that provides second focal spot to a target location on a patient, wherein the first focal spot is of a first type and produced with the first x-ray source at a first power and the second focal spot that is of a second type and produced with the second x-ray source at a second power;
one or more detectors that detects and records one or more images from the first focal spot and recording one or more images from the second focal spot; and
a computer system comprising a processor and computer-readable storable medium that stores instructions to combine the one or more images from the first focal spot the one or more images from the second focal spot to provide high-resolution multi-resolution data that is able to resolve fine features and with fluence to reduce noise levels and processes the high-resolution multi-resolution data using a model-based iterative reconstruction (MBIR) method.

12. The system of claim 11, wherein the first x-ray source and the second x-ray source are the same x-ray source or different x-ray sources.

13. The system of claim 12, wherein the first x-ray source and the second x-ray source comprises a dual-source, dual detector CT scanner, a single-source CT scanner with different tube settings, and x-ray tubes with adjustable focal spots.

14. The system of claim 11, wherein the first type and the second type comprise different sizes, different geometries, different number of focal spots, or combinations thereof.

15. The system of claim 11, wherein the model-based iterative reconstruction (MBIR) method uses a trained or untrained neural network.

16. The system of claim 11, wherein the model-based iterative reconstruction (MBIR) method processes the first focal spot and the second focal spot sequentially.

17. The system of claim 11, wherein the model-based iterative reconstruction (MBIR) method processes the first focal spot and the second focal spot concurrently.
